(19) United States
(12) Patent Application Publication
(10) Pub. No.: US 2007/0166743 A1
(43) Pub. Date: Jul. 19, 2007

(54) Quantum dots and methods of use thereof

(75) Inventor: Rudolf Gilmanshin, Waltham, MA (US)

Correspondence Address: WOLF GREENFIELD & SACKS, P.C., 600 ATLANTIC AVENUE, BOSTON, MA, 02210-2206, US

(73) Assignee: U.S. Genomics, Inc., Woburn, MA (US)

(21) Appl. No.: 11/634,412

(22) Filed: Dec. 6, 2006

Related U.S. Application Data
(63) Continuation of application No. 10/924,146, filed on Aug. 23, 2004, now abandoned.
(60) Provisional application No. 60/497,191, filed on Aug. 21, 2003.

(51) Int. Cl. C12Q 1/68

(52) U.S. Cl. 435/6; 977/924

(57) ABSTRACT

Quantum dots and methods of use thereof for labeling and analyzing polymers such as nucleic acid molecules are described herein.

### RELATED APPLICATIONS

This application claims priority to provisional patent application having Ser. No. 60/497,191, filed Aug. 21, 2003 and entitled “QUANTUM DOTS AND METHODS OF USE THEREOF”, the entire contents of which are incorporated by reference herein.

### FIELD OF THE INVENTION

The invention provides quantum dots and methods of use thereof for labeling and analyzing polymers such as nucleic acid molecules.

### BACKGROUND OF THE INVENTION

Coincident detection is a technique that allows two or more distinct labels to be detected simultaneously. A general scheme of a coincident detection technique is presented in FIG. 1. The Figure shows a mixture of different macromolecules, as represented by the solid-line different sized circles and ellipses. In order to analyze the mixture, two types of tags are mixed together, as represented by the solid-lined circles numbered 1 and 2. These tags can bind specifically to two different sites in the macromolecules and have different fluorescent groups associated with them. The tags find their corresponding sites and bind to them, after which the fluorescence of microscopic volumes of the mixture is analyzed. The volumes must be small enough that no more than a single macromolecule or tag can exist within it at any time. The measurement can be done using, for example, epi-fluorescent confocal microscope detection [1]. In this scheme, emission from a volume as small as 1 fentolitre (fl) can be measured at a time. At concentrations of 1 nM and below, events in which more than one molecule is present in the 1 fl volume at any given time are rare. For a measurement, a stationary volume can be illuminated (like in fluorescence correlation spectroscopy, [1]) or the sample mixture can be moved through illuminated volume (i.e., the illuminated volume can be within a microcapillary through which the solution is pumped [2-4]). In the former case, sample molecules move through the volume via diffusion. Excitation at several wavelengths and detection at several spectral regions can be used simultaneously to excite and detect emission of several different types of fluorophores at the same time [5]. Different tags have different fluorophores which emit in different spectral regions. FIG. 1 shows a representative example with two types of tags/fluorophores.

In a simple form of coincident detection, no fluorescence is detected when the illuminated volume contains no fluorophores. Fluorescence of a type 1 fluorophore is detected when the illuminated volume contains either a free type 1 tag or a macromolecule with bound type 1 tag. Fluorescence of a type 2 fluorophore is detected when the illuminated volume contains either a free type 2 tag or a macromolecule with bound type 2 tag. Concentrations of all components are kept sufficiently low to virtually eliminate the probability that a free type 1 and free type 2 tag will be present in the illuminated volume at the same time by chance. Therefore, fluorescence of both type 1 and 2 fluorophores detected at the same time indicates a macromolecule with both type 1 and type 2 tags bound thereto. Although not absolutely required, removal of excess unbound tags from the mixture after completion of the binding step (between the tags and the macromolecules) also decreases the proportion of accidental coincidences (i.e., the dual presence of free type 1 and free type 2 tags/fluorophores in the illuminated volume).

An example of a molecular system which can be effectively performed with a coincidence detection is presented in FIG. 2. The analyzed molecule may be a messenger RNA (mRNA) coding for a particular protein. Two different tags can be synthesized that each hybridize to a unique site on the mRNA. Those tags can be made of oligonucleotides, PNA or LNA, for example. The tags with different sequences can be conjugated to different directly or indirectly detectable labels. An example of a directly detectable label is a fluorophore. Thus, as an example, tags 1 and 2 may be conjugated to tetramethylrhodamine (TAMRA) and Cy5 fluorophores, respectively. Cellular extracts can be analyzed using this system. Living cells usually contain many copies of different mRNA molecules. The proportions of different mRNAs change with time and conditions. Using specially designed pairs of tags and coincidence detection, the presence of an mRNA of interest can be detected and in some instances its concentration can be estimated.

It is to be understood that although this embodiment involving mRNA and oligonucleotide tags is discussed further, the same strategy can be applied to many different systems. For example, an enzyme can be detected using a fluorescent substrate analog as tag 1 and a fluorescent antibody conjugate as tag 2.

Coincidence detection is a powerful technique which allows detection of molecules with two particular sites of interest, even in the presence of a large amount of other molecules [5; 6]. For successful application of coincidence detection, detectable labels (and/or the tags to which they are bound) must be present at sufficiently low concentration and their signal must be clearly discriminated from background noise. The latter condition is difficult to satisfy with single molecule detection where typically only several tens of photons are detected during the time the fluorophore is resident in the illuminated volume. Usually, a discrimination scheme is used to separate useful signal from background (e.g., only spikes exceeding a threshold level are counted as useful fluorophore signals). A threshold level must be set at a level higher than background intensity and lower than useful signal. It is difficult to determine this level for single molecule fluorophores because of noise and low signal intensity. The level is either too high and therefore excludes many useful signal spikes (photon bursts) leading to decreased sensitivity, or it is too low and therefore permits too much background noise leading to a high and therefore unacceptable proportion of accidental coincidences.

Another problem with coincidence detection is the intrinsic need for multiple color excitation and detection. Several lasers are needed for excitation of different fluorophores and several detectors are needed to detect signals in different spectral regions. Furthermore, an effective separation of multicolor excitation and emission peaks is also required and this usually requires the use of expensive optical filters and dichroic mirrors. The instant invention alleviates these and other limitations.

### SUMMARY OF THE INVENTION

The invention relates in some aspects to methods for analyzing polymers such as nucleic acids using quantum dots. In one aspect the invention is a method for identifying a property of a nucleic acid by labeling a nucleic acid with a quantum dot and a detectable label and detecting a signal from the quantum dot and the detectable label to thereby identify a property of the nucleic acid. The detectable label may be a directly detectable label or an indirectly detectable label. In one embodiment the detectable label is a fluorophore.

The invention in another aspect is a method for identifying a property of a polymer such as a nucleic acid by exciting a donor molecule to produce a first emission, and detecting the presence or absence of a second emission from an acceptor molecule, wherein when a polymer has a property the polymer causes the donor and acceptor molecule to be brought into physical proximity such that the first emission excites the acceptor molecule and produces the second emission and the polymer is identified as having the property when the second emission is detected. At least one of the donor molecule and acceptor molecule is a quantum dot.

In one embodiment the donor molecule is a quantum dot and the acceptor molecule is a fluorophore. In another embodiment the quantum dot is labeled with a first tag and the first tag specifically interacts with the polymer and identifies the property of the polymer. In another embodiment the fluorophore is attached to a second tag and the second tag specifically interacts with the polymer. Alternatively the quantum dot is labeled with a first tag and the first tag specifically interacts with the polymer and the fluorophore is attached to a second tag and the second tag specifically interacts with the polymer and identifies the property of polymer. Preferably the polymer is a nucleic acid.

These and other embodiments of the invention will be described in greater detail herein.

### BRIEF DESCRIPTION OF THE FIGURES

FIG. 1 is a general scheme of a coincident detection technique.

FIG. 2 is a representation of a molecular system which can be effectively performed with coincidence detection.

FIG. 3 is a representation of a method of the invention using quantum dots.

FIG. 4A shows excitation and emission spectra of a typical organic fluorophore (e.g., fluorescein) presented by dashed and continuous lines respectively.

FIG. 4B shows excitation and emission spectra of a typical quantum dot presented by dashed and continuous lines respectively.

FIG. 4C shows emission spectra of a quantum dot and a fluorophore.

FIG. 5A shows the excitation and emission spectra of a FRET pair consisting of fluorescein as the donor and TAMRA as the acceptor.

FIG. 5B shows excitation and emission spectra of a quantum dot/fluorophore pairing in which the quantum dot has a very narrow emission spectrum with no “tail” and, as a result, there is no donor emission in the acceptor spectral range.

It is to be understood that the Figures are not required for enablement of the invention.

### DETAILED DESCRIPTION OF THE DESCRIPTION

The invention relates to the use of quantum dots in identifying properties of polymers. Quantum dots are nanometer scale particles that absorb light, then quickly re-emit the light but in a different wavelength and thus color. The dots have optical properties that can be readily customized by changing the size or composition of the dots. Quantum dots are available in multiple colors and brightness, offered by either fluorescent dyes or semiconductor LEDs (light emitting diodes). In addition, quantum dot particles have many unique optical properties such as the ability to tune the absorption and emission wavelength by changing the size of the dot. Thus different-sized quantum dots emit light of different wavelengths. Quantum dots have been described in U.S. Pat. No. 6,207,392, and are commercially available from Quantum Dot Corporation.

Quantum dots are composed of a core and a shell. The core is generally composed of cadmium selenide (CdSe), cadmium telluride (CdTe), or indium arsenide (InAs). CdSe provides emission on the visible range, CdTe in the red near infrared, and InAs in the near infrared (NIR). The composition and the size of the spherical core determine the optical properties of the quantum dot. For instance, a 3 nm CdSe quantum dot produces a 520 nm emission, a 5.5 nm CdSe quantum dot produces a 630 nm emission, and intermediate sizes result in intermediate colors. The emission width is controlled by the size distribution.

The outer shell of a quantum dot protects the core, amplifies the optical properties, and insulates the core from environmental effects. It also provides a novel surface coating to link the particles to biomolecules, such as polymers. Biomolecules such as but not limited to antibodies, streptavidin, lectins, and nucleic acids can be coupled to the quantum dots. Traditional light sources such as lamps, lasers, and LEDs are exemplary excitation sources for quantum dots.

The quantum dots may be used in conjunction with a detectable label. The detectable label can be directly detectable (i.e., one that emits a signal itself). Alternatively, the detectable label can be indirectly detectable (i.e., one that binds to or recruits another molecule that is itself directly detectable, or one that cleaves a product to generate directly detectable substrates). Generally, the detectable label can be selected from the group consisting of an electron spin resonance molecule (such as for example nitroxyl radicals), a fluorescent molecule, a chemiluminescent molecule, a radioisotope, an enzyme, an enzyme substrate, a biotin molecule, an avidin molecule, a streptavidin molecule, a peptide, an electrical charge transferring molecule, a colloid gold nanocrystal, a ligand, a microbead, a magnetic bead, a paramagnetic particle, a chromogenic substrate, an affinity molecule, a protein, a peptide, a nucleic acid, a carbohydrate, an antigen, a hapten, an antibody, an antibody fragment, and a lipid. Exemplary detectable labels include radioactive isotopes such as p³² or H³, luminescent markers such as fluorochromes, optical or electron density markers, etc., biotin, digoxigenin, or epitope tags such as the FLAG or HA epitope, avidin and enzymes such as alkaline phosphatase, horseradish peroxidase, β-galactosidase, etc. Other labels include chemiluminescent substrates, chromogenic substrates, fluorophores such as fluorescein (e.g., fluorescein succinimidyl ester), TRITC, rhodamine, tetramethylrhodamine, R-phycoerythrin, Cy-3, Cy-5, Cy-7, Texas Red, Phar-Red, allophycocyanin (APC), etc. Those of ordinary skill in the art will be familiar with detectable labels and the appropriate selection thereof based on the teachings provided herein.

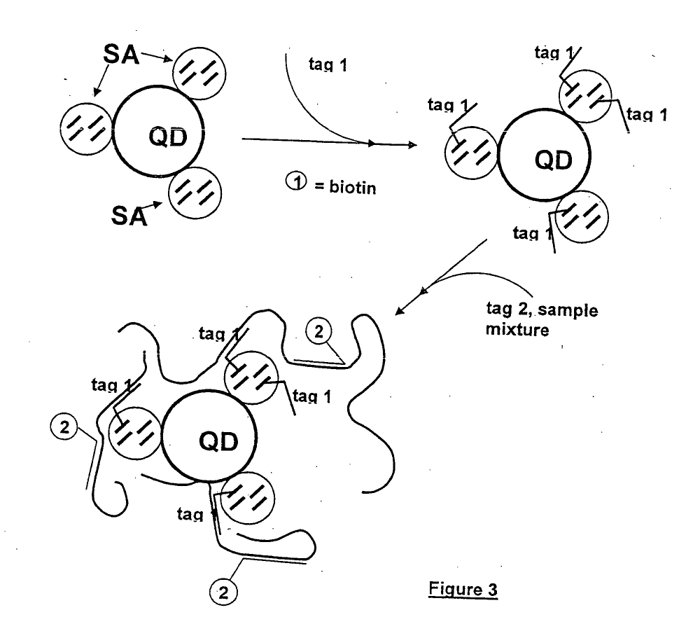

An example of a method of the invention using quantum dots is shown in FIG. 3. In this embodiment, commercially available quantum dots (QD) with multiple binding sites are used. In this particular example, quantum dots with multiple streptavidin (SA) molecules are used. Streptavidin is a protein that has four binding sites, each of which is capable of tightly and specifically binding to a biotin molecule. In the example, a first tag (tag 1) has a biotin group conjugated to it, and a second tag (tag 2) has a fluorophore conjugated to it. Both tags are oligonucleotides designed to hybridize to particular sites of a sample mRNA molecule (i.e., the target).

Tag 1 is first added to the solution of quantum dots with multiple SA. Biotin groups of the tag molecules are then bound to the SA binding sites. In general, more than one tag 1 molecule can bind to every SA molecule, and tag 1 molecules can bind to more than one SA of the quantum dots. Therefore, several tag 1 molecules can be bound to a quantum dot. Tag 2 and a sample are then mixed, and the mixture is analyzed. In another embodiment, all tags, samples, and quantum dots can be mixed in a single step. After mixing, mRNA molecules with appropriate hybridization sites bind to tag 1 molecules that are bound to the quantum dots. At the same time, tag 2 molecules bind to the appropriate sites of the mRNA molecules. As a result, a supramolecular complex is formed including a single quantum dot with several tag 2 molecules bound to it through the mRNA molecules.

Amplification. The accumulation of multiple tag 2 molecules results in a proportionally higher signal in the spectral region of the fluorophore attached thereto and thus allows higher efficiency of detection. Quantum dots also have a fluorescence intensity which is higher than that of organic fluorophores. Thus, in the example provided the emission in both spectral regions is much higher than in coincidence detection techniques which employ tags with single organic fluorophores. Higher intensities of both signals allows a more efficient threshold with a reliable cutoff of background signals. The higher signal intensity observed in this example is due to the use of quantum dots and to the amplification of fluorescent signals within the supramolecular assembly.

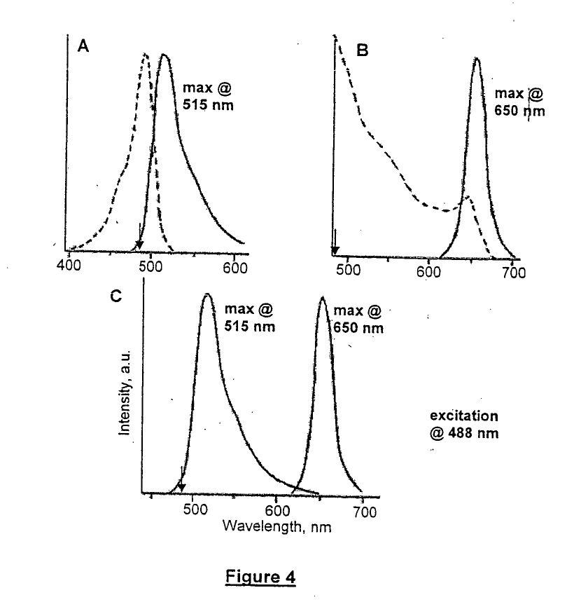

Single excitation source. One advantage of quantum dots is that they have a very wide excitation spectrum. In FIG. 4A, excitation and emission spectra of a typical organic fluorophore (in this case fluorescein) are presented by dashed and continuous lines respectively. These spectra are typical of organic fluorophores. First, the width of excitation spectrum (FWHH) (i.e., the range where an efficient excitation is possible) is 50-70 nm for organic fluorophores. Second, the Stokes shift (i.e., the distance between the maxima of excitation and emission spectra) is 10-25 nm for organic fluorophores. Third, the emission spectrum width (FWHH) is typically 50-80 mn. The arrow shows the position of 488 nm argon (Ar) laser wavelength which is typically used to excite fluorescein fluorescence. This laser line is conveniently very close to the maximum of the excitation spectrum. For coincidence detection, the emissions of different fluorophores should not overlap so as to detect each of them independently. Because the Stokes shift is smaller than the emission spectrum width, different organic fluorophores with emissions in sufficiently far spectral regions must be excited at different wavelengths. These wavelengths cut in between the emission spectra.

Quantum dots have distinctively different spectral properties. FIG. 4B depicts excitation (dashes) and emission (continuous) spectra of a typical quantum dot. The quantum dot excitation spectrum is wide. Moreover, its intensity gradually increases towards short wavelengths. Therefore, quantum dot fluorescence can be successfully excited in a very wide range of wavelengths and most preferably at shorter excitation wavelengths. A quantum dot emission spectrum is narrow—typically 30 nm FWHH. Fluorescence of this quantum dot can be effectively excited at 488 nm (the arrow in FIG. 4B).

Because the width of quantum dot excitation spectrum is much wider than the width of emission spectrum of an organic fluorophore, it is possible to find a pair of a quantum dot and an organic fluorophore having emission spectra that do not overlap, and thus to find a single wavelength which is capable of exciting fluorescence emission from both the organic fluorophore and quantum dot simultaneously (the arrow at 488 nm in FIG. 4C).

The combination of a quantum dot and an organic fluorophore according to the methods of the invention allows simultaneous detection of fluorescence (and therefore coincidence detection) using a single excitation wavelength. In other embodiment, a monochromator or a bandpass filter with a continuous spectrum light source can be used instead of a laser. In this case, the excitation light will include a range of wavelengths.

Spectral optimization. Spectral properties of organic fluorophores depend mostly on their chemical structure and to a lesser extent on their molecular surroundings and external conditions. Therefore, fluorophores must be specifically selected to correspond to a particular laser line and a tag design. There may be no appropriate laser line for some fluorophores. In contrast to organic fluorophores, the maximum quantum dot emission spectrum depends primarily on the size of the quantum dot. Therefore, a quantum dot with the most optimal spectral properties can be produced for any organic fluorophore. For example, CdSe quantum dots can be obtained with fluorescence spectrum maxima anywhere between 490 and 640 nm by varying their diameters. For other spectral ranges, quantum dots made of different materials can be used (i.e., CdTe quantum dots emit at wavelengths >670 nm). Spectral properties of the proposed molecular construct can always be optimized by adjusting the size and material of the quantum dot core to match the spectra of the selected organic fluorophore.

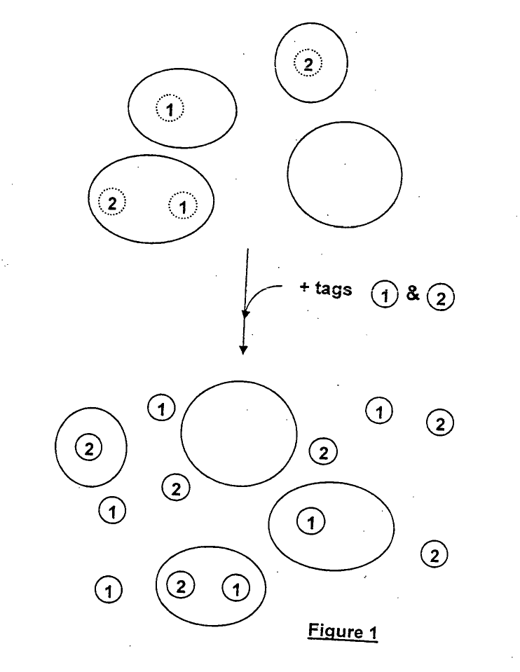

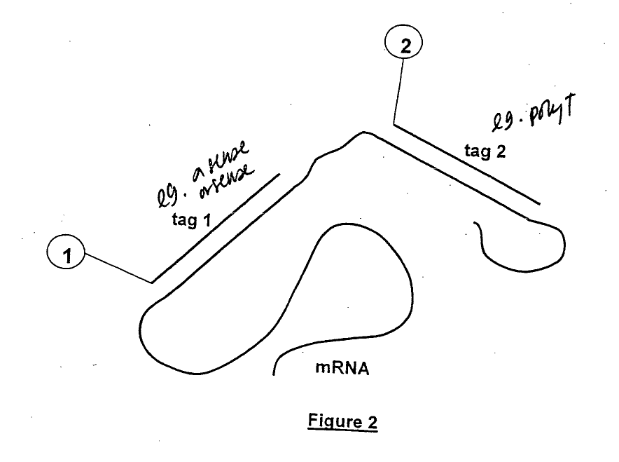

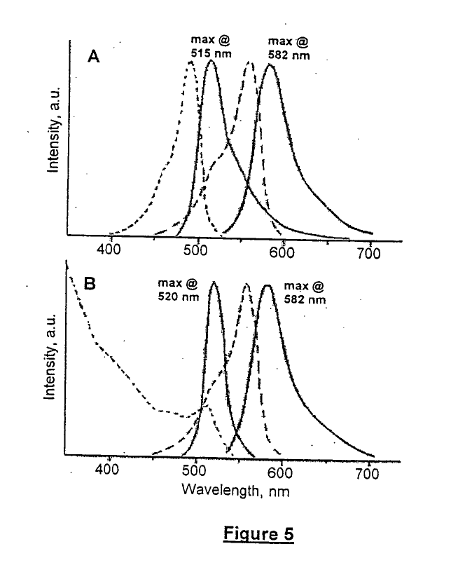

FRET—single detector detection. Information similar to coincidence detection can be obtained using fluorescence resonance energy transfer (FRET) [7]. In this case, fluorophores 1 and 2 constitute a donor-acceptor pair. An example of such a pair including fluorescein as a donor and TAMRA as an acceptor is presented in FIG. 5A. Fluorescein is excited close to the maximum of its excitation spectrum (short dashes). Its emission spectrum (continuous line, maximum @ 515 nm) overlaps with the excitation spectrum of TAMRA (long dashes). Therefore, the excitation energy from fluorescein can be transferred (donated) without direct radiation to TAMRA fluorophore (acceptor), from which it can be further emitted (continuous line spectrum with maximum @ 582 nm). This energy transfer can occur only through a very short distance (i.e., when both tag 1 and tag 2 are bound to the same molecule at a close proximity (FIGS. 1 and 2)). In a FRET scheme, the donor molecule is excited and fluorescence emission is detected from the acceptor molecule. Theoretically, the FRET approach has advantages over simple coincidence detection because it needs a single light source and a single detector, but in practice, it is difficult to implement.

Ideally, there should be no signal at all within the acceptor emission spectral range until the donor appears close to it. In this case, every detected photon would result from FRET and would indicate the formation of an interacting donor-acceptor pair. Such detection (on for example a “black” background) is very sensitive. However, organic fluorophores have wide spectra with long “tails” (FIG. 5A). The tails of the donor emission protrude into the acceptor emission window and as a result some photons detected are emitted by donor and indistinguishable from acceptor emission. The acceptor and donor can be excited by the same wavelength because of the tail of the excitation spectrum from the donor. In this case, the detected photons will be emitted by acceptor but due to its direct excitation instead of energy transfer. As a result, in addition to FRET both direct excitation of acceptor and direct emission of donor contribute to the detected signal. All those components have similar amplitudes; therefore, detection of a FRET signal is performed not on a “black” background but as a change of amplitude of non-zero emission. Because total number of photons emitted by single fluorophores is very low, such detection has very low sensitivity due to noise.

However, the FRET methods of the invention avoid these problems and can be successfully used for detection. In one example, the quantum dot, which is capable of interacting with or is labeled with a first tag (tag 1) serves as a donor and a second tag (tag 2) has an acceptor fluorophore conjugated to it (FIG. 5B). As a quantum dot has very narrow emission spectrum with no “tail,” there is no donor emission in the acceptor spectral range in this system. Because quantum dots have very wide excitation spectrums, they can be excited at shorter wavelengths (for example at 405 nm) where no direct excitation of the acceptor fluorophore occurs.

In the molecular assembly shown in FIG. 3, FRET is detected without interference of direct acceptor excitation or donor emission (i.e., on a “black” background) and therefore is very sensitive. Such detection methods can be performed with a single excitation light source and a single detector.

The sensitivity of methods provided herein allows single polymers such as nucleic acid molecules to be analyzed individually. The nucleic acid molecules may be single stranded and double stranded nucleic acids. Harvest and isolation of nucleic acid molecules are routinely performed in the art and suitable methods can be found in standard molecular biology textbooks (e.g., such as Maniatis' Handbook of Molecular Biology). The nucleic acid may be a DNA or an RNA. DNA includes genomic DNA (such as nuclear DNA and mitochondrial DNA), as well as in some instances cDNA. RNA includes mRNA but is not so limited. In important embodiments, the nucleic acid molecule is a genomic nucleic acid molecule. In related embodiments, the nucleic acid molecule is a fragment of a genomic nucleic acid molecule. The size of the nucleic acid molecule is not critical to the invention and it generally only limited by the detection system used.

The target molecule (i.e., the molecule being studied or analyzed) is generally a polymer, such as but not limited to a nucleic acid. The size of the target nucleic acid molecule is not limiting. It can be several nucleotides in length, several hundred, several thousand, or several million nucleotides in length. In some embodiments, the nucleic acid molecule may be the length of a chromosome.

The term “nucleic acid” is used herein to mean multiple nucleotides (i.e. molecules comprising a sugar (e.g. ribose or deoxyribose) linked to an exchangeable organic base, which is either a substituted pyrimidine (e.g. cytosine (C), thymidine (T) or uracil (U)) or a substituted purine (e.g. adenine (A) or guanine (G)). “Nucleic acid” and “nucleic acid molecule” are used interchangeably. As used herein, the terms refer to oligoribonucleotides as well as oligodeoxyribonucleotides. The terms shall also include polynucleosides (i.e., a polynucleotide minus a phosphate) and any other organic base containing polymer. Nucleic acid molecules can be obtained from existing nucleic acid sources (e.g., genomic or cDNA), or by synthetic means (e.g. produced by nucleic acid synthesis).

The methods of the invention use tags such as a nucleic acid tag molecule. As used herein, a nucleic acid tag molecule is a molecule that is able to recognize and bind to a specific nucleotide sequence within a target nucleic acid molecule (i.e., the nucleic acid molecule intended to be labeled and/or analyzed).

It is to be understood that any nucleic acid analog that is capable of recognizing a nucleic acid molecule with structural or sequence specificity can be used as a nucleic acid tag molecule. In most instances, the nucleic acid tag molecules will form at least a Watson-Crick bond with the nucleic acid molecule. In other instances, the nucleic acid tag molecule can form a Hoogsteen bond with the nucleic acid molecule, thereby forming a triplex with the target nucleic acid. A nucleic acid sequence that binds by Hoogsteen binding enters the major groove of a nucleic acid target and hybridizes with the bases located there. Examples of these latter tag molecules include molecules that recognize and bind to the minor and major grooves of nucleic acids (e.g., some forms of antibiotics). In preferred embodiments, the nucleic acid tag molecules can form both Watson-Crick and Hoogsteen bonds with the target nucleic acid molecule. BisPNA tag molecules are capable of both Watson-Crick and Hoogsteen binding to a nucleic acid molecule. In most embodiments, tag molecules with strong sequence specificity are preferred.

Preferably, the nucleic acid tag molecules recognize and bind to sequences within the target polymer (i.e., the polymer being labeled and/or analyzed). If the polymer is itself a nucleic acid molecule, then the nucleic acid tag molecule preferably recognizes and binds by hybridization to a complementary sequence within the target nucleic acid. The specificity of binding can be manipulated based on the hybridization conditions. For example, salt concentration and temperature can be modulated in order to vary the range of sequences recognized by the nucleic acid tag molecules.

The length of the tag molecule (and the target sequence) determines the specificity of binding. The energetic cost of a single mismatch between the tag molecule and the nucleic acid target is relatively higher for shorter sequences than for longer ones. Therefore, hybridization of small sequences is more specific than is hybridization of longer sequences because the longer sequences can embrace mismatches and still continue to bind to the target depending on the conditions. One potential limitation to the use of shorter tag molecules however is their inherently lower stability at a given temperature and salt concentration. In order to avoid this latter limitation, bisPNA tag molecules can be used which allow both shortening of the target sequence and sufficient hybrid stability in order to detect tag molecule binding to the nucleic acid molecule being analyzed.

Another consideration in determining the appropriate tag molecule length is whether the sequence to be detected is unique or not. If the method is intended only to sequence the target nucleic acid, then unique sequences may not be that important provided they are sufficiently spaced apart from each other to be able to detect the signal from each binding event separately from the others.

The nucleic acid molecules can be analyzed using the Gene Engine™ system described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system allows single nucleic acid molecules to be passed through an interaction station in a linear manner, whereby the nucleotides in the nucleic acid molecules are interrogated individually in order to determine whether there is a detectable label conjugated to the nucleic acid molecule. Interrogation involves exposing the nucleic acid molecule to an energy source such as optical radiation of a set wavelength. In response to the energy source exposure, the detectable label on the nucleotide (if one is present) emits a detectable signal. The mechanism for signal emission and detection will depend on the type of label sought to be detected.

### REFERENCES

1. Eigen M, Rigler R. “Sorting single molecules: application to diagnostics and evolutionary biotechnology.” Proc. Natl. Acad Sci. U.S.A. (1994) 91: 5740-5747.
2. Agronskaia A, Schins J M, De Grooth B G, Greve J. “Two-color fluorescence in flow cytometry DNA sizing: Identification of single-molecule fluorescent probes.” Anal. Chem. (1999) 71: 4684-4689.
3. Chou H-P, Spence C, Scherer A, Quake S R. “A microfabricated device for sizing and sorting DNA molecules.” Proc. Natl. Acad. Sci. U.S.A. (1999) 96: 11-13.
4. Foquet M, Korlach J, Zipfel W, Webb W W, Craighead H G. “DNA fragment sizing by single molecule detection in submicrometer-sized closed fluidic channels.” Anal. Chem. (2002) 74: 1415-1422.
5. Heinze K G, Rarbach M, Jahnz M, Schwille P. “Two-Photon Fluorescence Coincidence Analysis: Rapid Measurements of Enzyme Kinetics.” Biophys. J. (2002) 83: 1671-1681.
6. Winkler T, Kettling U, Koltermann A, Eigen M. “Confocal fluorescence coincidence analysis: An approach to ultra high-throughput screening.” PNAS (1999) 96: 1375-1378.
7. Didenko V, D. “DNA probes using fluorescence resonance energy transfer (FRET): Designs and applications.” BioTechniques (2001) 31: 1106-1121.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

What is claimed is:

1. A method for identifying a property of a nucleic acid comprising
labeling a nucleic acid with a quantum dot and a detectable label,
detecting a signal from the quantum dot and the detectable label to identify a property of the nucleic acid.

2. The method of claim 1, wherein the detectable label is a fluorophore.

3. A method for identifying a property of a polymer comprising
exciting a donor molecule to produce a first emission, and detecting the presence or absence of a second emission from an acceptor molecule, wherein when a polymer has a property the polymer causes the donor and acceptor molecule to be brought into physical proximity such that the first emission excites the acceptor molecule and produces the second emission and the polymer is identified as having the property when the second emission is detected, and wherein at least one of the donor molecule and acceptor molecule is a quantum dot.

4. The method of claim 3, wherein the donor molecule is a quantum dot.

5. The method of claim 4, wherein the acceptor molecule is a fluorophore.

6. The method of claim 5, wherein the quantum dot is labeled with a first tag and wherein the first tag specifically interacts with the polymer and identifies the property of the polymer.

7. The method of claim 6 wherein the fluorophore is attached to a second tag and wherein the second tag specifically interacts with the polymer.

8. The method of claim 5, wherein the quantum dot is labeled with a first tag and wherein the first tag specifically interacts with the polymer.

9. The method of claim 6, wherein the fluorophore is attached to a second tag and wherein the second tag specifically interacts with the polymer and identifies the property of polymer.

10. The method of claim 3, wherein the polymer is a nucleic acid.
